(19) United States
(12) Patent Application Publication
(10) Pub. No.: US 2025/0064719 A1
(43) Pub. Date: Feb. 27, 2025

(54) COSMETIC COMPOSITION FOR ANTI-AGING OR WRINKLE REDUCTION COMPRISING CULTURE BROTH OF NEURAL STEM CELLS AS ACTIVE INGREDIENT AND METHOD OF PREPARING SAME

(71) Applicant: KOREA UNIVERSITY RESEARCH AND BUSINESS FOUNDATION, Seoul (KR)

(72) Inventors: Sung Hoi HONG, Seoul (KR); In Sik HWANG, Seoul (KR)

(73) Assignee: KOREA UNIVERSITY RESEARCH AND BUSINESS FOUNDATION, Seoul (KR)

(21) Appl. No.: 18/939,509

(22) Filed: Nov. 6, 2024

Related U.S. Application Data
(63) Continuation of application No. 16/972,792, filed on Dec. 7, 2020.

(30) Foreign Application Priority Data
Jun. 5, 2018 (KR) 10-2018-0064704

(51) Int. Cl. A61K 8/98; A61K 8/02; A61K 8/04; A61K 8/64; A61K 8/66; A61Q 1/12; A61Q 19/00; A61Q 19/08; A61Q 19/10

(52) U.S. Cl. CPC A61K 8/981; A61K 8/0208; A61K 8/0212; A61K 8/046; A61K 8/64; A61K 8/66; A61Q 1/12; A61Q 19/007; A61Q 19/08; A61Q 19/10

(57) ABSTRACT

The present invention relates to a method of preparing a culture broth of neural stem cells, having excellent abilities of reducing skin wrinkles or increasing skin elasticity, the culture broth comprising high concentrations of TIMP-1 and TIMP-2 capable of inhibiting the expression and activity of matrix metalloproteinases (MMPs) involved in collagen decomposition in the dermis. The culture broth of neural stem cells according to the present invention, which comprises, as active ingredients, high concentrations of TIMP-1 and TIMP-2 and low concentrations of various MMPs, restores the synthesis of collagen and elastin by suppressing the expression and activity of MMPs inhibiting collagen formation, and thus is useful as a composition for reducing skin wrinkles or increasing skin elasticity.

### CROSS-REFERENCE TO RELATED APPLICATIONS

This application is a continuation of U.S. patent application Ser. No. 16/972,792 filed on Dec. 7, 2020, which is a U.S. National Stage Application of International Application No. PCT/KR2019/006578 filed on May 31, 2019, which claims the benefit under 35 USC 119(a) and 365(b) of Korean Patent Application No. 10-2018-0064704 filed on Jun. 5, 2018, in the Korean Intellectual Property Office, the entire disclosures of which are incorporated herein by reference for all purposes.

### TECHNICAL FIELD

The present invention relates to a method of preparing a neural stem cell conditioned medium that has excellent ability to reduce skin wrinkles or enhance skin elasticity and contains, as active ingredients, high concentrations of TIMP-1 (tissue inhibitor of metalloproteinase 1) and TIMP-2 (tissue inhibitor of metalloproteinase 2) and low concentrations of various MMPs, and more particularly to a method of preparing a neural stem cell conditioned medium containing, at high concentrations, TIMP-1 and TIMP-2, which can reduce the expression of various types of MMPs (matrix metalloproteinases) involved in collagen degradation in the dermis and inhibit the activity of the MMPs.

### BACKGROUND ART

The skin protects the human body from the external environment and is responsible for various physiological functions. The skin is largely composed of three layers: the epidermis, the dermis and the subcutaneous fat. The epidermis is mainly composed of cells that form keratin, namely keratinocytes, and cells that produce melanin pigment, namely melanocytes, and the dermis is mainly composed of connective tissue, containing collagen and elastin elastic fibers, and matrix, and includes muscles, hair follicles, blood vessels, nerves and the like.

The causes of skin aging include internal causes related to increasing age and external causes originating in the external environment. Collagen, which is closely related to the formation of wrinkles in the skin, is produced in fibroblasts of the skin, makes up 90% of the dermis, and is known to decrease with increasing age and upon application of external stimuli such as ultraviolet rays (S. D. Shapiro., Curr. Opin. Cell Biol. 10, 1996; Naylor E C et al., Maturitas, 2011). It is known that aging or continuous exposure to external ultraviolet rays increases the expression and activity of MMPs (matrix metalloproteinases) which degrade connective tissue including collagen, and promotes degradation of collagen in the skin dermis tissue, resulting in decreased collagen content (A. Mauviel, J. Cell. Biochem., 1993; T. Quan et al., J. Investig. Dermatol. Symp. Proc. 2009). Here, it is known that the expression of MMPs is regulated by actions of the transcription factors AP-1 and NFkB on the promoters of the genes for MMPs (G. J. Fisher & J. J. Voorhees., J. Investig. Dermatol. Symp. Proc., 1998; K. Abeyama et al., J. Clin. Invest. 2000). In addition, the balance of TIMP (tissue inhibitor of matrix metalloproteinase), which inhibits the activity of MMPs when maintaining homeostasis of collagen in tissues, is important.

TIMPs (tissue inhibitors of metalloproteinase) include TIMP-1, TIMP-2, TIMP-3 and TIMP-4, and act as inhibitors of MMPs present in vivo. Among them, TIMP-1 is known to bind to the pro form of 92 kDa MMP-9 and MMP-1 among type IV collagenase and thereby irreversibly inhibit MMP, and TIMP-2 is known to bind both to the pro form and to the active form of 72 kDa MMP-2, among type IV collagenase, and particularly to inhibit the active form of all MMPs (Y. A. Declerck et al., Biochem. J. 1993; W. Bode et al., Ann. N.Y. Acad. Sci. 1999).

In addition, elastin is known as an essential fibrous tissue involved in wrinkle formation, which is involved in skin elasticity along with collagen, and the degradation of elastin is known to be regulated by the effect of an enzyme called “elastase” (J. H. Chung et al., J. Invest. Dermatol., 117, 2001).

Accordingly, as a result of extensive efforts to develop a composition capable of alleviating and preventing skin wrinkles and improving skin elasticity, the present inventors have found that a neural stem cell conditioned medium composition, containing, at high concentrations, TIMP-1 and TIMP-2, which irreversibly inhibit the functions of various MMPs capable of inhibiting the synthesis of collagen in the skin and promoting decomposition thereof, and containing, at low concentrations, various MMPs, could be prepared by culturing neural stem cells, which are adult stem cells. Based on this finding, the present invention was completed.

### SUMMARY OF THE INVENTION

It is one object of the present invention to provide a method for preparing a neural stem cell conditioned medium that has improved ability to alleviate skin wrinkles or to enhance skin elasticity, and contains, at low concentrations, MMPs capable of inhibiting collagen production and contains, at high concentrations, TIMP-1 and TIMP-2, capable of restoring the synthesis of collagen and elastin by inhibiting the expression and activity of MMPs, and a cosmetic composition containing the neural stem cell conditioned medium.

To achieve the above objects, the present invention provides a method for preparing a neural stem cell conditioned medium having improved ability to alleviate skin wrinkles or to enhance skin elasticity, and containing TIMP-1 (tissue inhibitor of metalloproteinase 1) and TIMP-2 (tissue inhibitor of metalloproteinase 2) as active ingredients, the method including (a) immortalizing adult neural stem cells (NSCs) isolated from the ventricular zone of the brain, and (b) culturing the immortalized neural stem cells in a non-inducing medium to obtain a neural stem cell conditioned medium.

The present invention also provides a cosmetic composition for alleviating skin wrinkles or enhancing skin elasticity containing, as an active ingredient, a neural stem cell conditioned medium containing TIMP-1 and TIMP-2.

The present invention also provides a method for alleviating skin wrinkles or enhancing skin elasticity, using the neural stem cell conditioned medium containing TIMP-1 and TIMP-2.

The present invention also provides the use of the neural stem cell conditioned medium containing TIMP-1 and TIMP-2 for the alleviation of skin wrinkles and enhancement of skin elasticity.

The present invention also provides the use of the neural stem cell conditioned medium containing TIMP-1 and TIMP-2 for the preparation of a cosmetic composition for alleviating skin wrinkles or enhancing skin elasticity.

### DESCRIPTION OF DRAWINGS

FIG. 1 shows the result of analysis using WST-1, identifying that there is no cytotoxicity when human somatic cells are treated with a neural stem cell conditioned medium.

FIG. 2 shows the result of measurement using a fluorescence microplate reader, identifying the reduction of reactive oxygen species (ROS), increased due to UVB, upon treatment of UVB-exposed human somatic cells with the neural stem cell conditioned medium.

FIG. 3 shows the result of analysis using a fluorescent microscope, identifying the reduction of the reactive oxygen species (ROS), increased due to UVB, upon treatment of UVB-exposed human somatic cells with the neural stem cell conditioned medium.

FIG. 4 shows the result of analysis using qPCR, identifying that the expression of MMPs promoting the degradation of collagen (pro-collagen) due to UVB is inhibited upon treatment of UVB-exposed human somatic cells with the neural stem cell conditioned medium.

FIG. 5 shows the result of analysis using Western blotting, identifying that the expression of MMPs promoting the degradation of collagen (pro-collagen) due to UVB is inhibited upon treatment of UVB-exposed human somatic cells with the neural stem cell conditioned medium.

FIG. 6 shows the result of analysis using zymography, identifying that the expression of MMPs promoting the degradation of collagen (pro-collagen) due to UVB is inhibited upon treatment of UVB-exposed human somatic cells with the neural stem cell conditioned medium.

FIG. 7 shows that the collagen (pro-collagen), reduced due to UVB, is increased again upon treatment of UVB-exposed human somatic cells with the neural stem cell conditioned medium.

FIG. 8 shows that TIMP-1 and TIMP-2 inhibit the activity of MMPs in the neural stem cell conditioned medium.

FIG. 9 shows inhibition of activity of MMPs after blocking (neutralizing) the recombinant proteins, TIMP-1, TIMP-2, and neural stem cell conditioned medium using TIMP-1 and TIMP-2 antibodies.

FIG. 10 shows the result of visual observation, identifying that the wrinkles, increased due to UVB, are reduced again upon treatment of UVB-exposed female SKH-1 mice with the neural stem cell conditioned medium.

FIG. 11 shows the result of measurement of the area of the wrinkles, identifying that wrinkles, increased due to UVB, are reduced again upon treatment of UVB-exposed female SKH-1 mice with the neural stem cell conditioned medium.

FIG. 12 shows that ROS (reactive oxygen species), increased due to UVB, are reduced upon treatment of UVB-exposed female SKH-1 mice with the neural stem cell conditioned medium.

FIG. 13 shows that collagen and elastin in the dermis decreased due to UVB are increased again upon treatment of UVB-exposed female SKH-1 mice with the neural stem cell conditioned medium.

FIG. 14 shows the result of immunofluorescence staining, identifying that the expression of MMPs, which promote the degradation of collagen in the dermis due to UVB, is reduced upon treatment of UVB-exposed female SKH-1 mice with the neural stem cell conditioned medium.

FIG. 15 shows that the expression of MMPs, which promote the degradation of collagen in the dermis due to UVB, is inhibited upon treatment of UVB-exposed female SKH-1 mice with the neural stem cell conditioned medium.

FIG. 16 shows that the activity of MMPs, which promote the degradation of collagen in the dermis due to UVB, is inhibited upon treatment of UVB-exposed female SKH-1 mice with the neural stem cell conditioned medium.

FIG. 17 shows the result of identification that the neural stem cell conditioned medium is regulated through NFkB among the transcription factors that regulate the expression of MMPs.

FIG. 18 shows the result of identification that the production of collagen in the dermis is restored due to the decrease in transcription factors upon treatment of UVB-exposed female SKH-1 mice with the neural stem cell conditioned medium.

FIG. 19 shows the result of Western blotting, identifying that DNA damage caused by UVB is repaired through Rad50, one of the DNA repair enzymes, upon treatment of UVB-exposed female SKH-1 mice with the neural stem cell conditioned medium.

FIG. 20 shows the result of identification of DNA damage caused by UVB through γH2AX upon treatment of UVB-exposed female SKH-1 mice with the neural stem cell conditioned medium.

FIG. 21 shows the result of comparison in effects of inhibiting aging and wrinkle formation between the neural stem cell conditioned medium and the adipose-derived stem cell conditioned medium, wherein (a) shows the result of detection of cytotoxicity, and (b) shows the RNA expression levels of genes for MMPs upon treatment with each cell conditioned medium.

FIG. 22 (a) shows the result of comparison in the amount of MMP-1 released in each of the neural stem cell conditioned medium and the adipose-derived stem cell conditioned medium, and (b) shows the amount of MMP protein after treatment of UVB-exposed human somatic cells with each cell conditioned medium.

FIG. 23 shows the result of comparison in (a) total protein amount and (b) the amounts of TIMP-1 and TIMP-2 present in the neural stem cell conditioned medium and the adipose-derived stem cell conditioned medium.

FIG. 24 shows the result of (a) visual observation of the degree of inhibition of wrinkles formed due to UVB and (b) the wrinkle area measured upon treatment of UVB-exposed female SKH-1 mice with each of the neural stem cell conditioned medium and the adipose-derived stem cell conditioned medium.

FIG. 25 shows (a) the result of comparison of inhibition of expression of MMPs that promote the degradation of collagen in the dermis due to UVB and (b) the result of comparison of the production or restoration of collagen in the dermis upon treatment of UVB-exposed female SKH-1 mice with the neural stem cell conditioned medium and the adipose-derived stem cell conditioned medium.

FIG. 26 shows the result of a comparison of the degree of increase in collagen and elastin in the dermis, decreased due to application of UVB, upon treatment of UVB-exposed female SKH-1 mice with the neural stem cell conditioned medium and the adipose-derived stem cell conditioned medium.

FIG. 27 shows the result of immunofluorescence staining, identifying that the expression of MMPs that promote the degradation of collagen in the dermis due to UVB is reduced upon treatment of UVB-exposed female SKH-1 mice with the neural stem cell conditioned medium and the adipose-derived stem cell conditioned medium.

FIG. 28 shows (a) the result of Western blotting comparison in the repair of the DNA damage caused by UVB through Rad50, one of the DNA repair enzymes, and (b) and (c) the results of comparison in the levels of DNA damage based on γH2AX.

The patent or application file contains at least one drawing executed in color. Copies of this patent or patent application publication with color drawing(s) will be provided by the Office upon request and payment of the necessary fee.

### DETAILED DESCRIPTION AND PREFERRED EMBODIMENTS OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

In the present invention, human dermal fibroblasts and mice exposed to ultraviolet light (UVB) were treated with an ectoderm-derived neural stem cell conditioned medium obtained by culturing neural stem cells (NCSs) extracted from a ventricular zone of the human brain. Then, it was found that the expression and activity of MMP-2 (matrix metalloproteinase 2), MMP-9 (matrix metalloproteinase 9), and MMP-1 (matrix metalloproteinase 9), which are involved in the decomposition of collagen in the dermis in human dermal fibroblasts and mouse skin tissues exposed to ultraviolet light (UVB), were inhibited by the neural stem cell conditioned medium, and that the synthesis of collagen was restored or promoted by the inhibition of these MMPs. In addition, it was found that TIMP-1 and TIMP-2 were contained in the neural stem cell conditioned medium at high concentrations as active ingredients for alleviating skin wrinkles or enhancing skin elasticity through the recovery and promotion of collagen synthesis.

Therefore, in one aspect, the present invention is directed to a method for preparing a neural stem cell conditioned medium having improved ability to alleviate skin wrinkles or to enhance skin elasticity, and containing TIMP-1 (tissue inhibitor of metalloproteinase 1) and TIMP-2 (tissue inhibitor of metalloproteinase 2) as active ingredients, the method including (a) immortalizing adult neural stem cells (NSCs) isolated from the ventricular zone of the brain, and (b) culturing the immortalized neural stem cells in a non-inducing medium to obtain a neural stem cell conditioned medium.

In the present invention, there is no particular limitation as to types of the neural stem cells for preparing the cell conditioned medium. Preferably, the neural stem cells are adult stem cells derived from a fetus and, in a specific embodiment of the present invention, a neural stem cell conditioned medium is prepared using neural stem cells extracted from a ventricular zone of the fetal brain.

In the present invention, the neural stem cell conditioned medium is a substance containing a component released from cells obtained by subculturing neural stem cells, which are ectodermal stem cells.

In the present invention, the neural stem cell conditioned medium may further contain TIMP-3 or TIMP-4.

In the present invention, the neural stem cell conditioned medium inhibits the expression or activity of MMP-1 (matrix metalloproteinase-1), MMP-2 (matrix metalloproteinase-2), MMP-3 (matrix metalloproteinase-3) and MMP-9 (matrix metalloproteinase-9).

In a specific embodiment of the present invention, in order to determine the effect of the TIMP-1 and TIMP-2 components in the neural stem cell conditioned medium on alleviation of skin wrinkles and prevention or enhancement of skin elasticity, by inhibiting the expression and activity of MMPs (matrix metalloproteinases), the supernatant obtained from the neural stem cell conditioned medium was analyzed through cytokine array and western blot.

In addition, in a specific embodiment of the present invention, it was confirmed through gelatin zymography that the components TIMP-1 and TIMP-2 in the neural stem cell conditioned medium are involved in alleviation of wrinkle formation and enhancement of skin elasticity by inhibiting active MMPs.

In a specific embodiment of the present invention, treating with neural stem cell conditioned medium, recombinant protein TIMP-1 or TIMP-2 respectively or together, or blocking respectively or together through an antibody, and then binding to the pro form and the active form of 92 kDa MMP-9 and 72 kDa MMP-2 among type IV collagenases to inhibit the active form of MMPs was confirmed.

In the present invention, in order to confirm the effects of alleviating wrinkle formation and enhancing skin elasticity by the components of TIMP-1 and TIMP-2 in the neural stem cell conditioned medium, the backs of female SKH-1 mice were treated with UVB (30 mJ/cm) three times a week from 1 to 4 weeks, and then 200 μl (v/v) of PBS (phosphate buffered saline) and the neural stem cell conditioned medium was applied at 200 μl (v/v) to the backs of female SKH-1 mice. From 5 to 12 weeks, the backs of female SKH-1 mice were treated with UVB (30 mJ/cm), and PBS and neural stem cell conditioned medium were applied at 200 μl (v/v) thereto.

In the present invention, the neural stem cell conditioned medium may restore collagen or elastin in skin tissue.

In the present invention, the neural stem cell conditioned medium may inhibit the production of reactive oxygen species in skin tissue or repair DNA damage.

As used herein, the term “wrinkle alleviation” means maintaining or strengthening the ability relating to wrinkles and elasticity of the skin. Collagen fibers (collagen) and elastic fibers (elastin) present in the dermal layer of the skin structure are the main proteins that conduct these functions and are responsible for skin elasticity. The biosynthesis of collagen is affected internally and externally by the skin. Specifically, as an internal factor of the skin, natural aging causes a decrease in cellular activity and a decrease in collagen fibers, and as external factors thereof, reactive oxygen species produced by excessive irradiation of ultraviolet rays and stress react with the thiol group of proteins (thiol: —SH) to inhibit the activity of enzymes or to increase the expression of collagenase, which is an enzyme, namely matrix metalloproteinase-1 (MMP-1), which degrades collagen, elastin and the like, thereby increasing skin wrinkles and reducing skin elasticity.

As the medium for culturing neural stem cells of the present invention, any basal medium known in the art may be used without limitation. The basal medium may be prepared directly, or may be commercially available. Examples of commercially available media include, but are not limited to, DMEM (Dulbecco's Modified Eagle's Medium), MEM (Minimal Essential Medium), BME (Basal Medium Eagle), RPMI 1640, F-10, F-12, Q-MEM (α-Minimal essential Medium), G-MEM (Glasgow's Minimal Essential Medium) and Iscove's Modified Dulbecco's medium, and most preferred is a DMEM medium.

In addition, the basal medium preferably contains 5 to 10% (v/v) of FBS, and in a specific embodiment of the present invention, culture is conducted in DMEM medium.

In the present invention, the non-inducing medium may contain DMEM (Dulbecco's Modified Eagle's Medium), 10% FBS (fetal bovine serum), and 1% penicillin/streptomycin.

The composition of the present invention can be prepared using any commonly used method, and the process of isolating, culturing, and separating neural stem cells is not limited to the method of the present invention, and may be any commonly performed method.

As described above, the ectoderm-derived neural stem cell conditioned medium according to the present invention is capable of inhibiting expression of MMPs and active MMPs because it contains TIMP-1 and TIMP-2 as active ingredients and thus can be used as a composition for alleviating and preventing skin wrinkles, and improving skin elasticity.

Therefore, in another aspect, the present invention is directed to a cosmetic composition for alleviating skin wrinkles or enhancing skin elasticity containing the neural stem cell conditioned medium containing TIMP-1 (tissue inhibitor of metalloproteinase 1) and TIMP-2 (tissue inhibitor of metalloproteinase 2) as an active ingredient.

In the present invention, the neural stem cell conditioned medium may further contain TIMP-3 or TIMP-4.

In addition, the neural stem cell conditioned medium contains, at low concentrations, MMP-1 (matrix metalloproteinase-1), MMP-2 (matrix metalloproteinase-2), MMP-3 (matrix metalloproteinase-3), MMP-9 (matrix metalloproteinase-9), MMP-7 (matrilysin), MMP-10 (stromelysin) and MMP-13 (collagenase).

In the present invention, the neural stem cells are cells obtained by immortalizing neural stem cells extracted from a ventricular zone of the brain. In a specific embodiment of the present invention, the neural stem cells are obtained by immortalizing neural stem cells (NSCs) isolated from the ventricular zone of the fetal brain. The cells were cultured in a non-inducing medium containing DMEM (Dulbecco's Modified Eagle's Medium), 10% FBS (fetal bovine serum), and 1% penicillin streptomycin and non-adherent cells are removed. In the process of subculturing neural stem cells (NSCs) cultured through the above process, a neural stem cell conditioned medium is obtained and centrifuged, and the supernatant is filtered.

In the present invention, the neural stem cell conditioned medium inhibits the expression or activity of MMP-1 (matrix metalloproteinase-1), MMP-2 (matrix metalloproteinase-2), MMP-3 (matrix metalloproteinase-3) and MMP-9 (matrix metalloproteinase-9).

In the present invention, the neural stem cell conditioned medium may restore collagen or elastin in skin tissue.

In addition, in the present invention, the neural stem cell conditioned medium may inhibit the production of reactive oxygen species in skin tissue or repair DNA damage.

DNA damage repair is performed by increasing the activity of the DNA repair enzyme Rad50, Rad51 or XRCC4 by the neural stem cell conditioned medium. In addition, in the neural stem cell conditioned medium, gammaH2AX is used as a marker to determine the degree of DNA damage.

In the present invention, the term “cosmetic composition” refers to a composition containing the neural stem cell conditioned medium or a neural stem cell extract, and there is no particular limitation as to the formulation of the composition. For example, the formulation may be selected from the group consisting of skin lotion, skin softener, skin toner, astringent, lotion, milk lotion, moisture lotion, nutrition lotion, massage cream, nutrition cream, moisture cream, hand cream, foundation, essence, nutrition essence, pack, soap, cleansing foam, cleansing lotion, cleansing cream, body lotion, body cleanser, face wash, treatment, beauty liquids, beauty packs, ointments, gels, liniments, liquids/solutions, patches, and sprays.

In order to achieve the object of the present invention, the composition may be prepared and commercialized in the form of any of these formulations, and is not limited to the examples described above.

When formulated as a cosmetic composition, the content of the neural stem cell conditioned medium is 0.0001 to 50% by weight, preferably 0.01 to 10% by weight, based on the total weight of the cosmetic composition. In order to achieve the effect of minimizing or alleviating skin damage caused by ultraviolet rays, the content of the neural stem cell conditioned medium is preferably greater than the above minimum and is not greater than the above maximum in consideration of the possibility of application to various formulations and reduction of a sensation of excessive amount of addition. At this time, the content of the nerve cell culture conditioned medium is preferably properly adjusted within the above range according to the content of the ingredients contained in the formulation or cosmetic composition.

The ingredients included in the cosmetic composition of the present invention include ingredients commonly used in cosmetic compositions in addition to the active ingredient, for example, conventional adjuvants such as antioxidants, stabilizers, solubilizers, vitamins, pigments and fragrances, and carriers.

When the formulation of the present invention is a paste, cream or gel, animal oil, vegetable oil, wax, paraffin, starch, tragacanth, cellulose derivative, polyethylene glycol, silicone, bentonite, silica, talc, or zinc oxide may be used as a carrier component.

When the formulation of the present invention is a powder or spray, lactose, talc, silica, aluminum hydroxide, calcium silicate or polyamide powder may be used as a carrier component. In particular, when the formulation of the present invention is a spray, propellants such as chlorofluorohydrocarbon, propane/butane, or dimethyl ether may be further included.

When the formulation of the present invention is a solution or emulsion, a solvent, a solubilizing agent or an emulsifying agent, such as water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan, is used as a carrier component.

When the formulation of the present invention is a suspension, examples of useful carrier components include liquid diluents such as water, ethanol or propylene glycol, suspending agents such as ethoxylated isostearyl alcohol, polyoxyethylene sorbitol ester and polyoxyethylene sorbitan ester, and microcrystalline cellulose, aluminum metahydroxide, bentonite, agar, tragacanth and the like.

When the formulation of the present invention is a surfactant-containing cleanser, examples of useful carrier components include aliphatic alcohol sulfate, aliphatic alcohol ether sulfate, sulfosuccinic acid monoester, isethionate, imidazolinium derivatives, methyltaurate, sarcosinate, fatty acid amide ether sulfates, alkylamidobetaines, fatty alcohols, fatty acid glycerides, fatty acid diethanolamides, vegetable oils, lanolin derivatives, ethoxylated glycerol fatty acid esters and the like.

In another aspect, the present invention is directed to a method for alleviating skin wrinkles or enhancing skin elasticity, using the neural stem cell conditioned medium containing TIMP-1 and TIMP-2.

In another aspect, the present invention is directed to the use of the neural stem cell conditioned medium containing TIMP-1 and TIMP-2 for the alleviation of skin wrinkles and enhancement of skin elasticity.

In another aspect, the present invention is directed to the use of the neural stem cell conditioned medium containing TIMP-1 and TIMP-2 for the preparation of a cosmetic composition for alleviating skin wrinkles or enhancing skin elasticity.

### EXAMPLE

Hereinafter, the present invention will be described in more detail with reference to the following examples. However, it will be obvious to those skilled in the art that the following examples are provided only for illustration of the present invention, and should not be construed as limiting the scope of the present invention based on the subject matter of the present invention.

### Example 1: Production of Neural Stem Cell Conditioned Medium and Neural Stem Cell Extract

Cells were obtained by immortalizing adult neural stem cells (NSCs) isolated from the ventricular zone of the fetal brain.

Specifically, 14-week-old fetal nerve cell tissue was treated with 0.1% collagenase and 0.1% hyaluronidase solution at 37° C. for 1 hour and then treated with 0.05% trypsin-EDTA for 2 to 3 minutes to isolate individual cells. The individual cells were separated by FACS using a CD45−/CD133+/CD34− marker. The cells were cultured in human neurosphere culture medium supplemented with N-2 supplements, 0.2 mg/ml of heparin, 20 ng/ml of bFGF (basic fibroblast growth factor), 20 ng/ml of EGF (epidermal growth factor) and 10 ng/ml of LIF. After 10-14 days, the formed neurospheres were treated with a collagenase to separate the same into individual cells, and cells obtained by transduction of v-myc genes using a retroviral vector and selection were cultured in a non-induction medium containing DMEM (Dulbecco's Modified Eagle's Medium), 10% FBS (fetal bovine serum) and 1% penicillin/streptomycin (Flax J. D. et al., Nature Biotechnology, 16, 1998; Lim H-C et al., Neuroscience Letters 435:175-180, 2008). 5×10⁵ cells were seeded onto 150 mm culture dishes, 15 ml of a culture medium was added thereto, and the cells were cultured in a 37° C., 5% CO₂ incubator to obtain a culture broth when cell confluence reached 80%. The culture medium used herein was a non-induction medium containing DMEM (Dulbecco's Modified Eagle's Medium), 10% FBS (fetal bovine serum), and 1% penicillin streptomycin, and non-adherent cells were removed after culture. In the process of subculturing neural stem cells (NSCs) cultured in this way, a neural stem cell condition medium was obtained and centrifuged, and the supernatant was filtered.

### Example 2: Confirmation of Cytotoxicity of Neural Stem Cell Conditioned Medium to Human Somatic Cells

5×10³ human somatic cells were seeded onto a 96-well plate and treated with the neural stem cell conditioned medium for 24 hours when cell confluence reached 80 to 90%. WST-1 was added thereto, absorbance was measured at 450 nm using a microplate reader, and the cytotoxicity of the neural stem cell conditioned medium was detected.

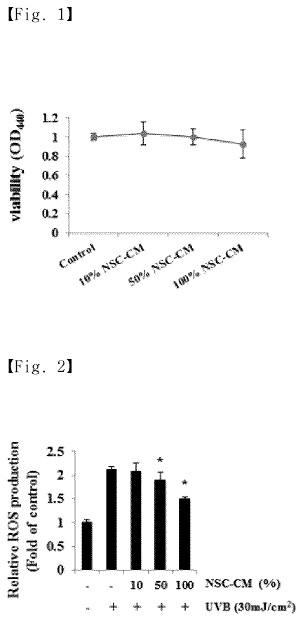

The result showed that the neural stem cell conditioned medium had no effect on proliferation of human somatic cells (FIG. 1).

### Example 3: Confirmation of Inhibition of Intracellular Reactive Oxygen Species Increased Due to UVB by Neural Stem Cell Conditioned Medium

5×10³ human somatic cells were seeded onto a black 96-well plate and cultured in serum-free DMEM for 24 hours when cell confluence reached 80 to 90%. Then, the human somatic cells were treated with the neural stem cell conditioned medium for 24 hours and exposed to UVB (30 mJ/cm²), and were then cultured in DMEM medium supplemented with 10% serum for 24 hours. In order to determine whether or not the neural stem cell conditioned medium inhibits the intracellular reactive oxygen species (ROS) increased due to UVB, addition of a 10 μM dihydroethidium (DHE) solution and treatment therewith at 37° C. for 30 minutes was conducted, followed by measurement using a fluorescence microplate reader.

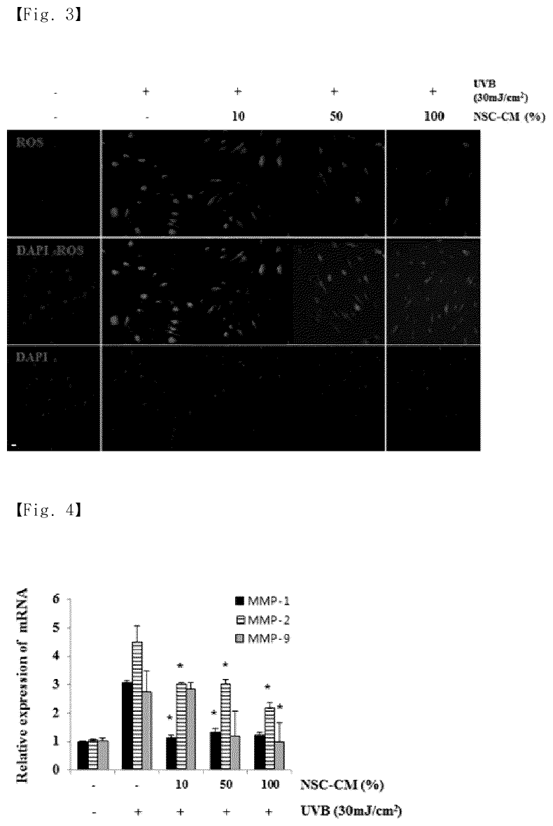

The result showed that that the neural stem cell conditioned medium is effective in reducing the intracellular reactive oxygen species increased due to UVB in human somatic cells (FIG. 2).

In addition, human somatic cells were seeded on a 24-well plate using a cover slip and cultured in serum-free DMEM for 24 hours, when the cell confluence reached 80 to 90%. Then, the human somatic cells were treated with the neural stem cell conditioned medium for 24 hours, exposed to UVB (30 mJ/cm²), and then cultured in DMEM supplemented with 10% serum for 24 hours. In order to confirm the effect of the neural stem cell conditioned medium on the intracellular reactive oxygen species (ROS) increased due to UVB, addition of a 10 μM dihydroethidium (DHE) solution thereto and analysis using a fluorescent microscope were conducted.

The result shows that the neural stem cell conditioned medium is effective in reducing intracellular reactive oxygen species (ROS) increased due to UVB in human somatic cells (FIG. 3).

### Example 4: Confirmation of Inhibition of MMPs Expression and Activity in Human Somatic Cells by Neural Stem Cell Conditioned Medium

When human somatic cells are exposed to UVB, degradation of collagen (pro-collagen) is promoted. Thus, in this example, the expression and activity of MMPs, which promote the degradation of collagen (pro-collagen), was confirmed.

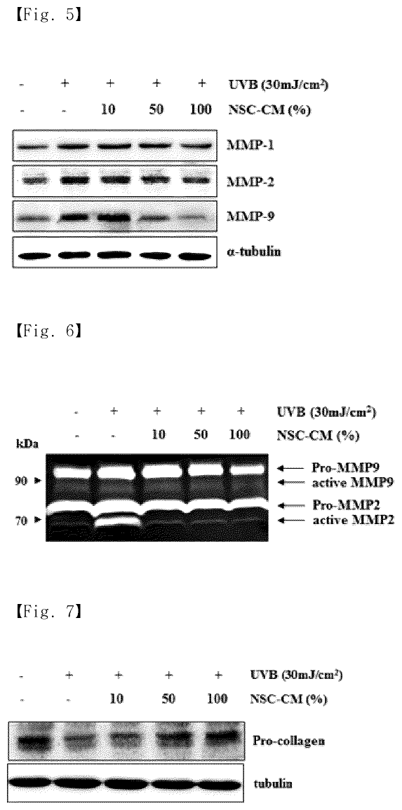

Human somatic cells were seeded onto a 100 mm culture dish and cultured in serum-free DMEM for 24 hours, when cell confluence reached 80 to 90%. Then, the human somatic cells were treated with the neural stem cell conditioned medium for 24 hours, exposed to UVB (30 mJ/cm²), and then cultured in DMEM medium supplemented with 10% serum for 24 hours. The effect of the neural stem cell conditioned medium on the expression and activity of MMPs, which promote the degradation of collagen (pro-collagen) upon exposure to UVB was determined through qPCR (FIG. 4), Western blot (FIG. 5), and zymography (FIG. 6).

The result shows that the neural stem cell conditioned medium inhibits the expression and activity of MMPs, which function to promote the degradation of collagen (pro-collagen) due to UVB (FIGS. 4 to 6).

### Example 5: Confirmation of Inhibition of Collagen Degradation Due to UVB by Neural Stem Cell Conditioned Medium

It was seen from Example 4 that, when human somatic cells are exposed to UVB, degradation of collagen (pro-collagen) is promoted and MMPs are involved in promoting the degradation of collagen (pro-collagen) in this way. Therefore, it was seen from this example that the synthesis of collagen (pro-collagen) was promoted by inhibiting the expression and activity of MMPs through the neural stem cell conditioned medium.

Cells were prepared in the same manner as in Example 4 and treated with the neural stem cell conditioned medium for 24 hours to inhibit the expression and activity of MMPs involved in promoting collagen degradation caused by UVB. Then, the synthesis of pro-collagen upon the inhibition of the expression and activity of MMPs by the neural stem cell conditioned medium was observed using Western blot.

The result shows that, when the neural stem cell conditioned medium inhibits the expression and activity of MMPs, involved in collagen degradation facilitated by UVB, the synthesis of collagen (pro-collagen) is promoted (FIG. 7).

### Example 6: Identification of Active Ingredients (Cytokines) TIMP-1/TIMP-2 in Neural Stem Cell Conditioned Medium Involved in Inhibition of MMP Activity

Human somatic cells were seeded onto a 150 mm culture dish, and were cultured with serum-free DMEM when cell confluence reached 80 to 90%. The cells were cultured for 48 hours to obtain a cell conditioned medium, a sample was prepared so that the amount of protein was 200 μg, and then the ingredients in the neural stem cell conditioned medium were detected using a cytokine array, specifically a human cytokine antibody array from RayBiotech.

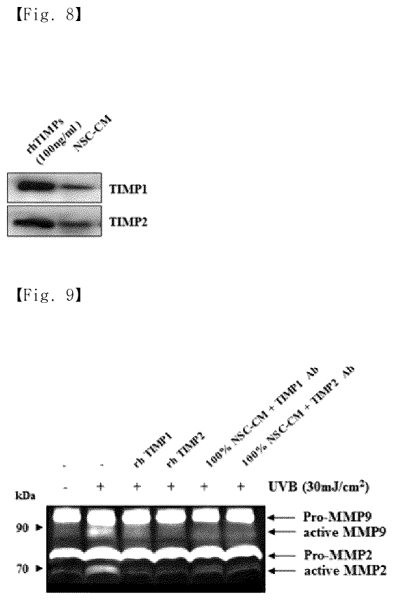

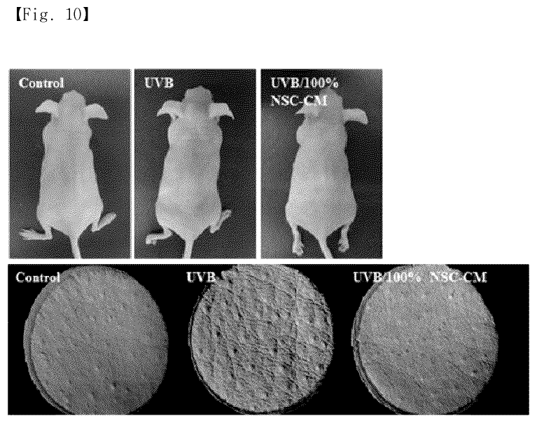

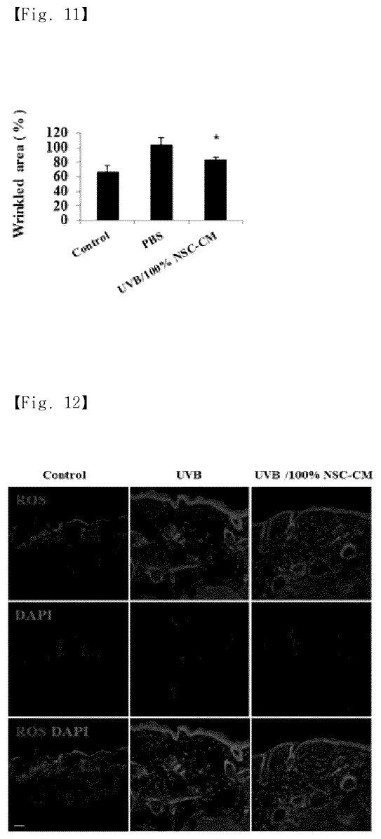

It was confirmed through the human cytokine antibody array and then again by Western-blot analysis (FIG. 8) that TIMP-1 and TIMP-2 were present in the neural stem cell conditioned medium.

Then, human somatic cells were seeded onto a 100 mm culture dish and cultured in serum-free DMEM for 24 hours when cell confluence reached 80 to 90%. Then, the neural stem cell conditioned medium was treated with each of the recombinant proteins TIMP-1 and TIMP-2 or a combination thereof for 24 hours, and human somatic cells were exposed to UVB (30 mJ/cm²) and cultured in DMEM medium supplemented with 10% serum for 24 hours. Whether or not the neural stem cell conditioned medium inhibits the activity of MMPs, promoted due to UVB, was determined.

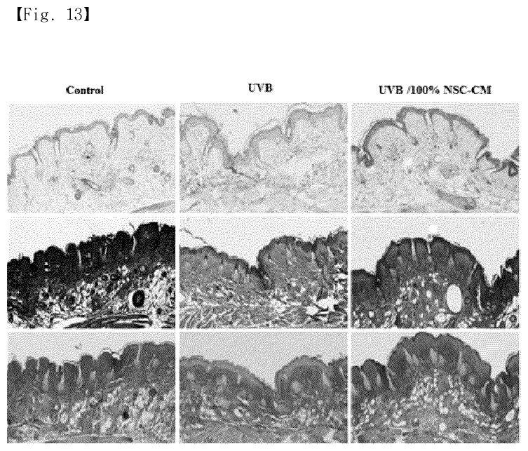

The result showed that the neural stem cell conditioned medium containing TIMP-1 and TIMP-2 inhibited the increased activity of MMPs attributable to UVB (FIG. 9).

### Example 7: Confirmation of Inhibition of Wrinkle Formation in SKH-1 Mice by Neural Stem Cell Conditioned Medium

### 7-1: Effect of Inhibiting Wrinkle Formation

Female SKH-1 mice were exposed to UVB (30 mJ/cm²/1 exposure) for 12 weeks, treated with the neural stem cell condition medium and then sacrificed at 12 weeks. The effects of inhibiting wrinkle formation and alleviating wrinkles by the group treated with the neural stem cell conditioned medium was analyzed through silicone replica.

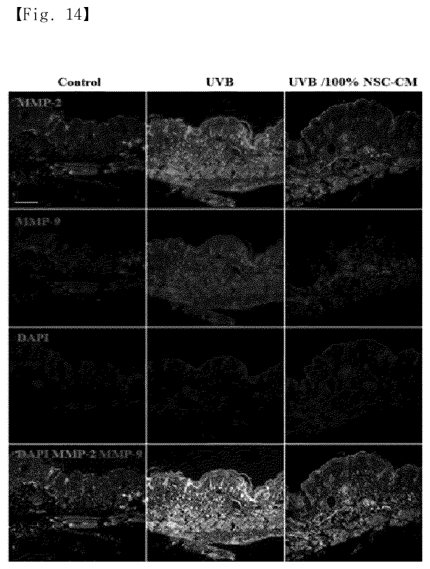

The result showed that the mice treated with the neural stem cell conditioned medium exhibited effects of inhibiting and preventing wrinkle formation and alleviating wrinkles (FIGS. 10 and 11).

### 7-2: Effect of Reducing ROS (Reactive Oxygen Species)

A tissue was prepared in the same manner as in Example 7-1 and the effect of the neural stem cell conditioned medium on ROS (reactive oxygen species) produced by UVB was analyzed by DHE staining.

The result showed that the effect of reducing ROS (reactive oxygen species) promoted by UVB in the mice treated with the neural stem cell conditioned medium was obtained (FIG. 12).

### 7-3: Effect of Recovering Collagen and Elastin

A tissue was prepared in the same manner as in Example 7-1, the mouse skin tissue was fixed in 4% formaldehyde, a tissue-embedded paraffin was produced and cut to a thickness of 3 μm, and the cut tissue was attached to the slide. Then, the behaviors of collagen and elastin decreased due to UVB radiation were detected through Masson's Trichrome staining and Verhoeff's elastin staining.

The result showed that collagen and elastin, decreased due to UVB radiation, were recovered in the mice treated with the neural stem cell conditioned medium (FIG. 13).

### Example 8: Confirmation of Reduction of MMP-2 and MMP-9 in SKH-1 Mice by Neural Stem Cell Conditioned Medium

### 8-1: Reduction of MMP-2 and MMP-9 Increased Due to UVB

A tissue was prepared in the same manner as in Example 7-1, the mouse skin tissue was fixed in 4% formaldehyde, a tissue-embedded paraffin was produced and was cut to a thickness of 3 μm, the cut tissue was attached to the slide, and deparaffinization and rehydration were performed. Then, antigen retrieval (citrate buffer, pH 6.0), treatment with 0.1% Triton X-100, blocking with normal donkey serum, and treatment with primary antibody (1:100) for 24 hours were sequentially performed. Then, the tissue was treated with a secondary antibody for 1 hour and subjected to DAPI staining for 5 minutes. The tissue was fixed to a slide using a vectashield mounting medium and was observed with a confocal microscope.

The result showed that MMP-2 and MMP-9, increased due to UVB, were reduced in the mice treated with the neural stem cell conditioned medium (FIG. 14).

8-2: Transcription Factor NFkB that Regulates MMPs Increased Due to UVB

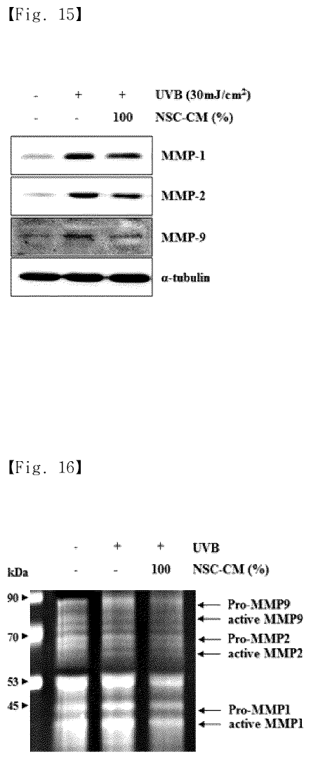

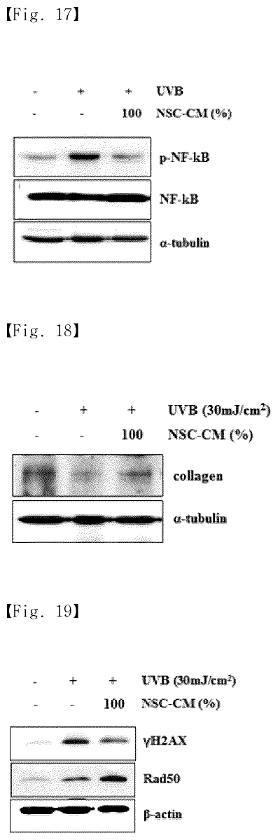

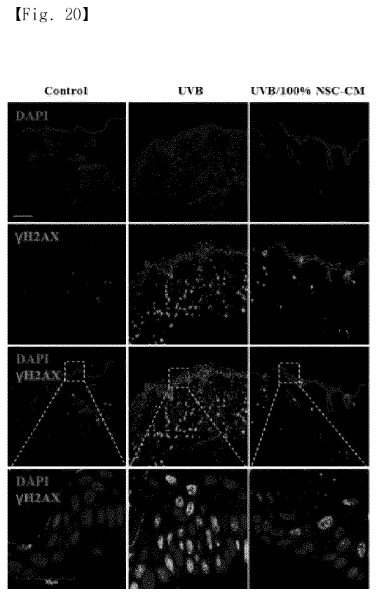

A tissue was prepared in the same manner as in Example 7-1, and expression and activity of MMPs involved in the degradation of intradermal collagen promoted by UVB in mouse skin tissue and expression of transcription factors that regulate the MMPs were detected by Western blot (FIGS. 15 to 17). In addition, the effect of the neural stem cell conditioned medium on collagen production through MMPs and transcription factors regulating the MMPs was investigated (FIG. 18).

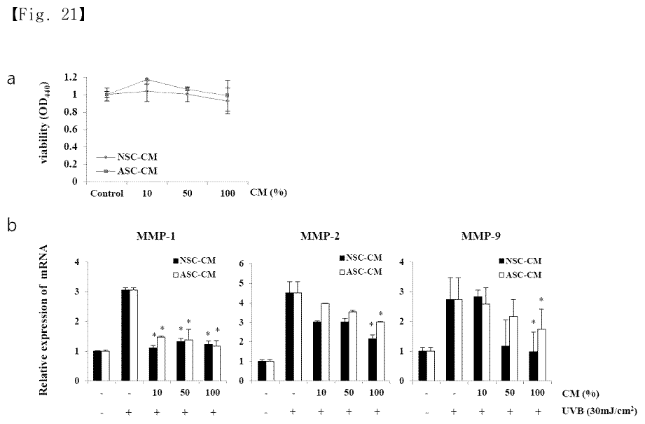

The result showed that the expression (FIG. 15) and activity (FIG. 16) of MMP-1, MMP-2 and MMP-9, increased due to UVB radiation, were reduced in the mice treated with the neural stem cell conditioned medium. In addition, it was found that NFkB, a transcription factor regulating these MMPs, was also reduced (FIG. 17) and thus collagen in the dermis was recovered again (FIG. 18).

### Example 9: Confirmation of DNA Damage Inhibition and Recovery by Neural Stem Cell Conditioned Medium

A tissue was prepared in the same manner as in Example 7-1, and was stained in the same manner as in Example 8-1, and the degree of DNA damage caused by UVB was determined using γH2AX. In addition, in order to determine the effect of the neural stem cell conditioned medium on DNA damage, Rad50, one of the DNA repair enzymes, was detected through Western blot.

The result showed that DNA damage caused by UVB was repaired in the mice treated with the neural stem cell conditioned medium and the damaged DNA was repaired through Rad50, one of the DNA repair enzymes (FIGS. 19 and 20).

### Example 10: Production of Neural Stem Cell Conditioned Medium and Adipose-Derived Stem Cell Conditioned Medium

For the preparation of neural stem cell conditioned medium (NSC-CM) and adipose-derived stem cell conditioned medium (ASC-CM), 5×10⁵ cells were seeded on 150 mm culture dishes, 15 ml of a culture medium was added thereto, and the cells were cultured in an incubator at 37° C. and 5% CO₂ to obtain a culture broth when the cell confluence reached 80%. The culture medium for the neural stem cells used herein was DMEM (Dulbecco's Modified Eagle's Medium) supplemented with 10% FBS (fetal bovine serum) and 1% penicillin streptomycin, and the culture medium for adipose-derived stem cells used herein was a non-inductive medium containing DMEM/F12 (Dulbecco's Modified Eagle's Medium: Nutrient Mixture F-12), 10% FBS (fetal bovine serum) and 1% penicillin streptomycin. After culture, non-adherent cells were removed. In the process of subculturing the neural stem cells (NSCs) and adipose-derived stem cells (ASCs) cultured through this process, a cell conditioned medium was obtained and centrifuged, and the resulting supernatant was filtered. In addition, it was found through WST-1 that each cell conditioned medium was not cytotoxic (FIG. 21a).

### Example 11: Comparison of Inhibition of Expression of MMPs In Vitro by Neural Stem Cell Conditioned Medium and Adipose-Derived Stem Cell Conditioned Medium

### 11-1: Identification of Expression of MMPs

When human somatic cells are exposed to UVB, the degradation of collagen (pro-collagen) is promoted. Therefore, in this example, expression of MMPs that promote the degradation of collagen (pro-collagen) was identified, and MMP-1 present in each stem cell conditioned medium was identified.

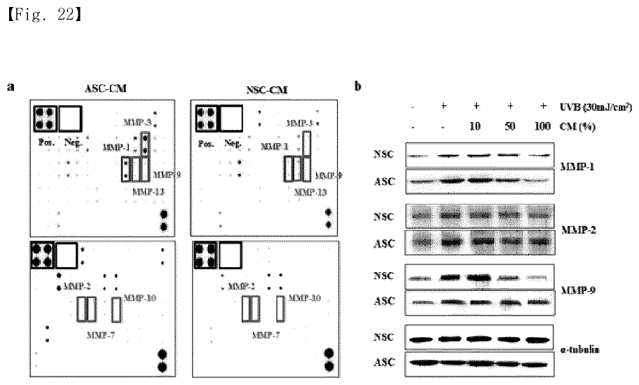

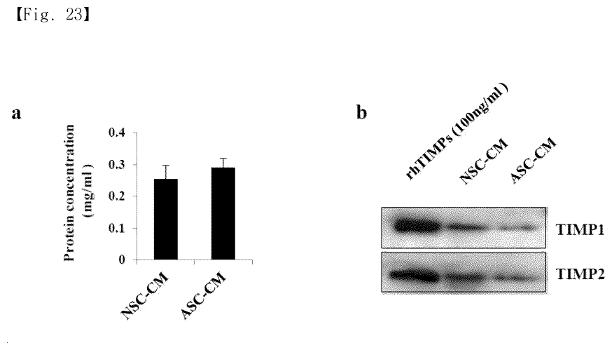

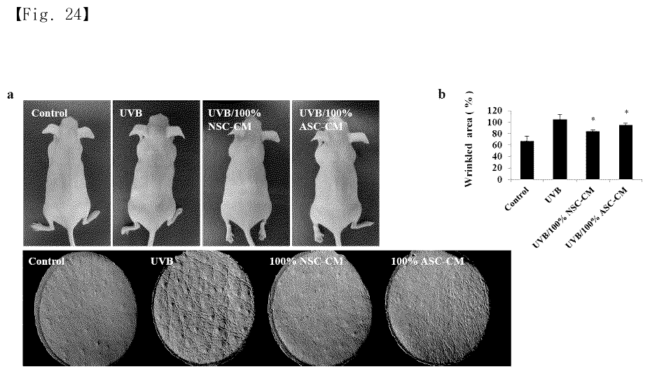

Human somatic cells were seeded onto a 100 mm culture dish, cultured with serum-free DMEM when cell confluence reached 80 to 90%, and were further cultured with serum-free DMEM for 24 hours. Then, the cells were treated with the neural stem cell conditioned medium and the adipose-derived cell conditioned medium for 24 hours, and the human somatic cells were exposed to UVB (30 mJ/cm²) and cultured in DMEM medium supplemented with 10% serum for 24 hours. The effect of the neural stem cell conditioned medium and the adipose-derived stem cell conditioned medium on the expression of MMPs, which promote the degradation of collagen (pro-collagen) upon exposure to UVB, was determined using qPCR (FIG. 21b) and Western blot (FIG. 22b).

The result showed that the expression of MMPs, which function to promote the degradation of collagen (pro-collagen) upon UVB radiation, was greater inhibited in the neural stem cell conditioned medium.

### 11-2: Comparison Between Factors Relating to Inhibition of Wrinkle Formation

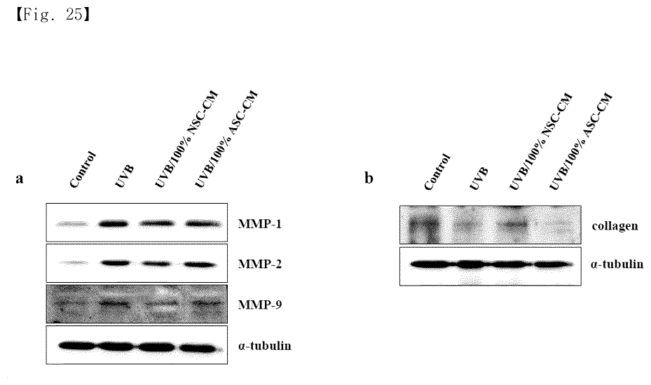

In this example, it was identified whether or not MMPs that promote the degradation of collagen (pro-collagen) and factors (TIMP-1 and TIMP-2) that inhibit the activity of the MMPs are present in the neural stem cell conditioned medium and the adipose-derived stem cell conditioned medium. The total protein amount in each conditioned medium was detected through Brad-ford assay (FIG. 23a), and then the concentrations of MMP-1, MMP-2, MMP-3, MMP-7, MMP-9, MMP-10, and MMP-13 were detected through the RayBio® Human cytokine antibody array (FIG. 22a). In addition, after treatment with each condition medium, the expression of MMPs was analyzed through Western blot (FIG. 23b).

The result showed that the neural stem cell conditioned medium had much lower content of MMP-1, MMP-2, MMP-3 and MMP-9, which promote collagen degradation, and higher content of TIMP-1 and TIMP-2, which inhibit the activity of MMPs, than that of the adipose-derived stem cell conditioned medium. Moreover, the result showed that the content of other MMPs, namely MMP-7, MMP-10 and MMP-13, was similar between the respective conditioned media. Therefore, it can be predicted that the neural stem cell conditioned medium has better ability to protect collagen due to the lower content of major MMPs therein.

### Example 12: Comparison of the Effects of Neural Stem Cell Conditioned Medium and Adipose-Derived Stem Cell Conditioned Medium In Vivo

### 12-1: Confirmation of Inhibition of Wrinkle Formation in SKH-1 Mice by Neural Stem Cell Conditioned Medium and Adipose-Derived Stem Cell Conditioned Medium

Female SKH-1 mice were exposed to UVB for 12 weeks (30 mJ/cm², 3 times per week over 1-4 weeks, 30 mJ/cm², once per week, over 5-12 weeks), treated with neural stem cell conditioned medium, and sacrificed at 12 weeks. The effects of inhibiting wrinkle formation and alleviating wrinkles by the groups treated with the neural stem cell conditioned medium and the adipose stem cell culture medium were analyzed through silicone replica.

The result showed that the effects of inhibiting wrinkle formation and alleviating wrinkles by the mice treated with the neural stem cell conditioned medium was significantly improved compared to the case of the adipose-derived stem cell conditioned medium (FIGS. 25a and 25b).

### 12-2: Effect of Recovering Collagen and Elastin in SKH-1 Mice by Neural Stem Cell Conditioned Medium and Adipose-Derived Stem Cell Conditioned Medium

A tissue was prepared in the same manner as in Example 7, the mouse skin tissue was fixed in 4% formaldehyde, a tissue-embedded paraffin was produced and was cut to a thickness of 3 μm, and the cut tissue was attached to the slide. Then, collagen and elastin, decreased due to UVB radiation, were analyzed through Masson's Trichrome staining and Verhoeff's elastin staining.

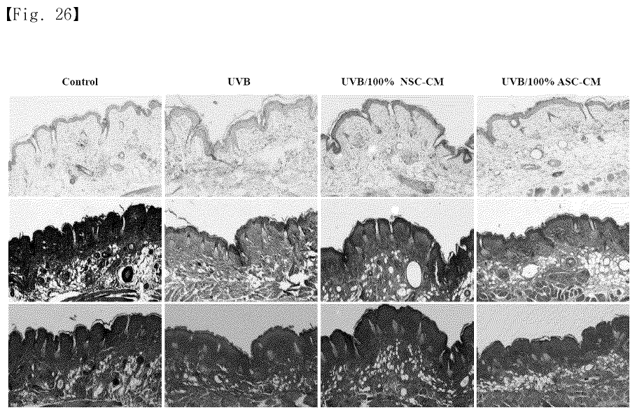

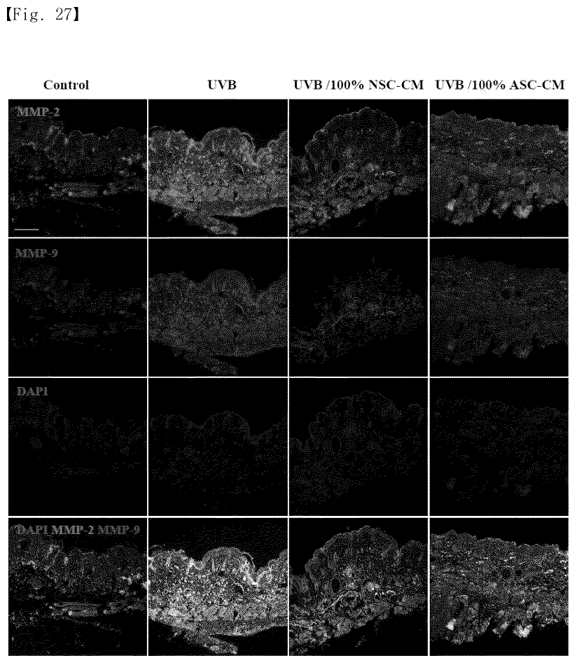

The result shows that collagen and elastin, decreased due to UVB radiation, were recovered more in the mice treated with the neural stem cell conditioned medium than in the mice treated with the adipose-derived stem cell conditioned medium (FIG. 26).

### Example 13: Comparison of Inhibition of Expression of MMPs In Vivo by Neural Stem Cell Conditioned Medium and Adipose-Derived Stem Cell Conditioned Medium

A tissue was prepared in the same manner as in Example 7-1, the mouse skin tissue was fixed in 4% formaldehyde, a tissue-embedded paraffin was produced and cut to a thickness of 3 μm, the cut tissue was attached to the slide, and deparaffinization and rehydration were performed. Then, antigen retrieval (citrate buffer, pH 6.0), treatment with 0.1% Triton X-100, blocking with normal donkey serum, and treatment with primary antibody (1:100) for 24 hours were sequentially performed. Then, the tissue was treated with a secondary antibody for 1 hour and subjected to DAPI staining for 5 minutes. The tissue was fixed to the slide using a vectashield mounting medium and was observed with a confocal microscope.

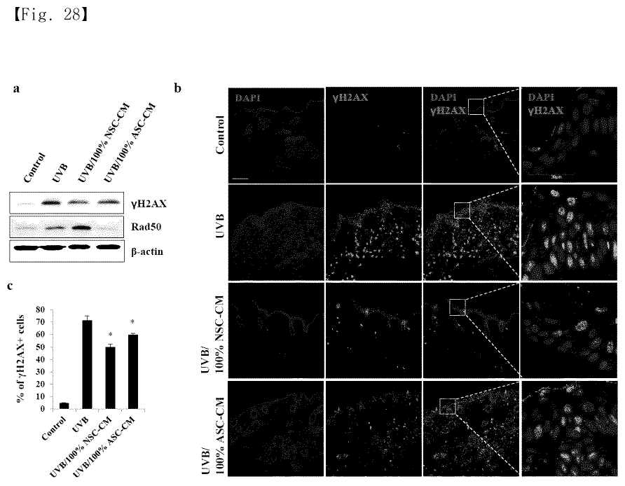

The result showed that MMP-2 and MMP-9, increased due to UVB was decreased more in the mice treated with the neural stem cell conditioned medium than the mice treated with the adipose-derived stem cell conditioned medium (FIG. 27).

### Example 14: Confirmation of DNA Damage Inhibition and Recovery by Neural Stem Cell Conditioned Medium and Adipose-Derived Stem Cell Conditioned Medium

A tissue was prepared in the same manner as in Example 7-1 and was stained in the same manner as in Example 8-1, and the degree of DNA damage caused by UVB was determined by γH2AX. In addition, in order to determine the effects of the neural stem cell conditioned medium and the adipose-derived stem cell conditioned medium on DNA damage, Rad50, one of the DNA repair enzymes, was detected through Western blot.

The result showed that DNA damage caused by UVB was repaired more in the mice treated with the neural stem cell conditioned medium than in the mice treated with the adipose-derived stem cell conditioned medium and the damaged DNA was repaired more through Rad50, one of the DNA repair enzymes (FIG. 28).

### INDUSTRIAL APPLICABILITY

The neural stem cell conditioned medium containing low concentrations of various MMPs and high concentrations of TIMP-1 and TIMP-2 as active ingredients according to the present invention inhibits the expression and activity of MMPs inhibiting collagen production, to thereby restore the synthesis of collagen and elastin, and thus is useful as a composition for alleviating skin wrinkles and improving skin elasticity.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

1. A method for inhibiting production of reactive oxygen species or for repairing DNA damage in skin tissue comprising:
immortalizing adult neural stem cells (NSCs) isolated from a ventricular zone of the brain;
obtaining a neural stem cell culture medium by culturing the immortalized neural stem cells in a non-inductive medium; and
treating the neural stem cell culture medium to the skin tissue,
wherein the neural stem cell culture medium comprises TIMP-1 (tissue inhibitor of metalloproteinase 1) and TIMP-2 (tissue inhibitor of metalloproteinase 2) as active ingredients, and
wherein the skin tissue comprises the reactive oxygen species produced by ultraviolet ray B or the DNA damage caused by ultraviolet ray B.

2. The method of claim 1, wherein the neural stem cell culture medium inhibits expression or activity of MMP-1 (matrix metalloproteinase-1), MMP-2 (matrix metalloproteinase-2), MMP-3 (matrix metalloproteinase-3) and MMP-9 (matrix metalloproteinase-9).

3. The method of claim 1, wherein the repairing the DNA damage is performed by increasing expression of Rad50.

4. The method according to claim 1, wherein the non-inducing medium comprises DMEM (Dulbecco's Modified Eagle's Medium), 10% FBS (fetal bovine serum), and 1% penicillin/streptomycin.
